(19) United States
(12) Patent Application Publication
(10) Pub. No.: US 2010/0281982 A1
(43) Pub. Date: Nov. 11, 2010

(54) Hearing Test and Screening System and Its Method

(76) Inventor: Wen-Huei LIAO, Taipei City (TW)

Correspondence Address: Guice Patents PLLC, 12647 Galveston Court #302, Manassas, VA, 20112, US

(21) Appl. No.: 12/436,974

(22) Filed: May 7, 2009

(51) Int. Cl. A61B 5/12

(52) U.S. Cl. 73/585

(57) ABSTRACT

A hearing test and screening system for testing and screening the hearing acuity of a person which includes a computer having built therein a graphic-control program for generating a pure tone signal required for a hearing test, an audio apparatus having a connection port coupled to the computer for receiving the pure tone signal from the computer and outputting a set of test signals, each having 3 frequencies minimum, at a constant sound volume upon receipt of the pure tone signal and a response button for operation by the test person to provide a feedback signal to the computer, and earphones coupled to the audio apparatus for enabling the test person to hear the set of test signals outputted by the audio apparatus.

### BACKGROUND OF THE INVENTION

1. Field of the Invention

The present invention relates to a method and system of testing and screening for hearing impairment and more particularly, to a hearing test and screening system having the characteristics of high portability and high hearing acuity measurement accuracy, simple hearing screening procedure and short hearing screening test time. The invention relates also to a hearing test and a system for hearing screening.

2. Description of the Related Art

Unidentified hearing-loss patients include patients with unilateral and mild sensorineural or conductive hearing loss, i.e., congenital hearing loss, otitis media effusion, sudden sensorineural hearing loss, noise induced hearing loss, cancer patient under chemotherapy or radiation therapy, Meniere's disease or autoimmune diseases. These patients may not notice or know their hearing loss condition, resulting in a delay in seeking for treatment, i.e., the so-called delayed hearing loss diagnosis. However, the hearing loss conditions of these patients exist constantly, causing silent potential injury to the patients or their families and producing communication barriers. If young children grow up with hearing loss, a delayed diagnosis of the hearing loss will affect the development in language, body and mind of these children. It is not easy to diagnose patients with potential hearing loss early. This work needs, on one hand, to enhance people's concern about hearing care and health education on hearing protection, and on the other hand, to have hearing examination be popularized. Routine hearing test or hearing screening is the most simple and effective way to find the unidentified patients with hearing loss.

There are numerous reasons indicate that why hearing test cannot be popularized at present, including: 1. Local out-patient clinics and medical center hospitals can not perform a regular hearing test immediately, and most patients need to make an appointment in advance, so that patients usually spend a lot of time waiting; 2. Most people do not understand the meanings of hearing tests and test results so that the importance of hearing test is usually neglected; 3. Setting up a complete hearing test instrument is rather expensive that includes the cost of hearing test equipments, a standard soundproof room, and profession manpower for the operation of hearing tests; therefore there are little out-patient clinics can assess patients' hearing acuity.

International Electrotechnical Commission defined five different types of audiometers according to their functioning: 1. Type I audiometer (i.e. pure-tone audiometers for professional diagnosis) is a dual-channel design with two independent systems to facilitate operation in the frequency range from 125 Hz to 10 kHz wherein, the maximum of air conduction output volume is 120 dBHL, the maximum of bone conduction output volume is 70 dBHL, having the wideband and narrowband masking noise sounds and capable of adding noise sound to the same side or opposite side air-conduction earphone as well as bone-conduction vibrator. These audiometers use a meter or LCD screen to display frequency and dB values, providing with 1˜5 dB hearing level stepwise shifts and many other test functions. Except the basic pure-tone air conduction and bone conduction hearing tests, a Type I audiometer can also carry out numerous types of hearing tests, including the speech test, sound-field test, short increment sensitivity index test, differential threshold value test, sound attenuation test and alternate binaural loudness balance test.

2. Type II audiometers (diagnostic type audiometers) which is a dual-channel design having the frequency range 125 Hz to 8 kHz wherein the maximum of air conduction output volume is 110 dBHL and the maximum of bone conduction output volume is 60 dBHL; capable of producing continuous pure-tone or pulse tone; having amplitude modulation means; providing masking white noise and narrowband sounds. An audiometer of this type can be used for oral sound, sound disc or magnetic type sound tape speech tests, short increment sensitivity index analysis, and differential threshold value test.

3. Type III audiometers (simple diagnostic audiometers), i.e., the monitoring or portable audiometers in the frequency range 250 Hz to 8 kHz, wherein the maximum of air conduction output volume is 100 dBHL, the maximum of bone conduction output volume is 50 dBHL and with or without masking noise sound. An audiometer of this type has a small size convenient for carrying, and therefore it is suitable for use in fundamental units or for medical attendant use.

4. Type IV audiometers (screening audiometers) that are classified into two categories, i.e., the group screening audiometers and the portable screening audiometers in the frequency range 250 Hz˜4 kHz to 6 kHz with air conduction but without bone conduction, wherein the sound volume range is 0 to 70/90 dBHL. Some fourth type audiometers provide simply four frequencies within the range 500 Hz to 4 kHz and four sound volume levels including 20 dBHL, 30 dBHL, 40 dBHL and 60 dBHL. An audiometer of this fourth type is particularly suitable for quick screening and group monitoring. Further, an audiometer of this fourth type provides a response button for clicking by the test person.

5. Type V audiometers (simple type audiometers). This type is simple in frequency range and hearing sound volume range without any minimum requirements. An audiometer of this fifth type allows setting of the frequency and hearing sound volume subject to demand, however it has no response button.

Further, existing standard professional hearing examinations are mainly to examine the hearing power. Except qualitative and quantitative hearing power examination, a standard professional hearing examination is preferably capable of discovering potential signs at an early stage. However, the known existing audiometers require professional speech and hearing manpower to operate and calibrate the equipment.

Taiwan Patent Application No. 095117076 discloses a fully automatic audiometer, which comprises: a sound generating module for generating sound subject to a predetermined measurement frequency and measurement sound volume, a feedback module adapted for providing a signal indicative of the sound received, and a control module. After the control module instructed the sound generating module to generate sound and the feedback module informed the control module of its receipt of the sound signal from the sound generating module, the control module records the measurement frequency and the measurement sound volume in a database module. This patent application simply provides one measurement frequency and one measurement sound volume and records the data in a database module after the measurement is done. After the test, the test person needs to return to the hospital or medical facility to hear professional medical staff's explanation about the test result. Before knowing the test result, the test person may be unable to keep an ease feeling. Further, this design is a so-called automatic design; however it cannot read the test result and is unable to diagnose hearing loss.

Therefore, it is desirable to provide a simple, rapid system for hearing test and screening that eliminates the aforesaid drawbacks.

### SUMMARY OF THE INVENTION

The present invention has been accomplished under the circumstances in view. It is one object of the present invention to provide a hearing test and screening system, which is highly portable and, provides high hearing acuity measurement accuracy.

It is another object of the present invention to provide a hearing test and screening system, which facilitates the operation of the hearing screening procedure.

It is still another object of the present invention to provide a hearing test and screening system, which shortens the hearing screening time and, immediately gives the test result to the test person.

It is still another object of the present invention to provide a hearing test and screening system, which provides threshold values to the test person for reference.

It is still another object of the present invention to provide a hearing test and screening system, which runs programmable test for routine hearing screening.

To achieve these and other objects of the present invention, a hearing test and screening system for testing and screening the hearing acuity of a person according to the present invention comprises a computer having built-therein a graphic-control program for generating a pure tone signal required for a hearing test, an audio apparatus, which comprises a connection port connected to the computer for enabling the audio apparatus to receive the pure tone signal from the computer and to output a set of test signals, each comprising at least 3 frequencies, at a constant sound volume upon receipt of the pure tone signal and a response button operable by the test person to provide a feedback signal to the computer, and earphones coupled to the audio apparatus for enabling the test person to hear the set of test signals outputted by the audio apparatus.

To achieve these and other objects of the present invention, a hearing test and screening method for testing and screening the hearing acuity of a person according to the present invention comprises the steps of: (a) setting a normal minimum measurement threshold value as an initial hearing test sound volume and providing a set of test signals, each test signal comprising at least 3 frequencies, and adjusting the normal minimum measurement threshold value upwards/downwards at a range of a predetermined amount of dBHL each time; (b) reducing a specific value from the normal minimum measurement threshold value and then running the test again at the reduced value if the sound volume at the normal minimum measurement threshold value passed the test; (c) reducing the specific value from the reduced value and then running the test at the secondarily reduced value if the reduced value passed the test, and then adding the specific value to the reduced value and running the test again if the sound volume at the secondarily reduced value did not pass the test; and (d) regarding the sound volume dBHL value that passed the test twice in three tests to be the average minimum measure threshold value that represents the hearing test and screening result.

### BRIEF DESCRIPTION OF THE DRAWINGS

FIG. 1 is a schematic drawing showing a hearing test and screening system in accordance with the present invention.

FIG. 2a is a schematic drawing showing the first application program of the hearing test and screening system according to the present invention.

FIG. 2b is a hearing screening report diagram of the second application program of the hearing test and screening system according to the present invention.

FIG. 3 is a comparison of the hearing screening report diagram of the invention and a conventional visual acuity chart.

FIG. 4 is a hearing test and screening flow chart according to the present invention.

### DETAILED DESCRIPTION OF THE PREFERRED EMBODIMENT

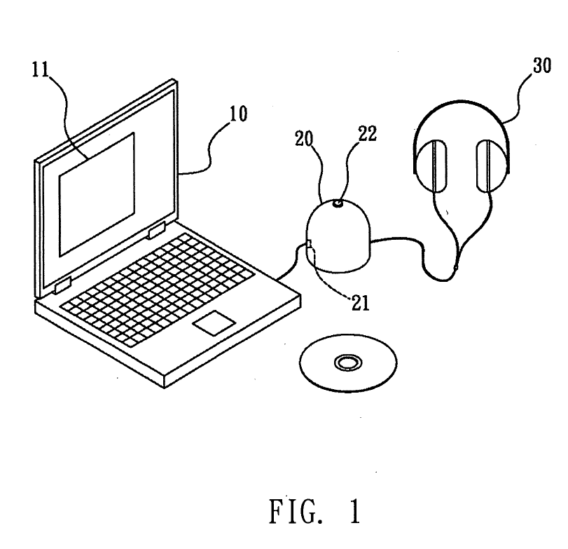

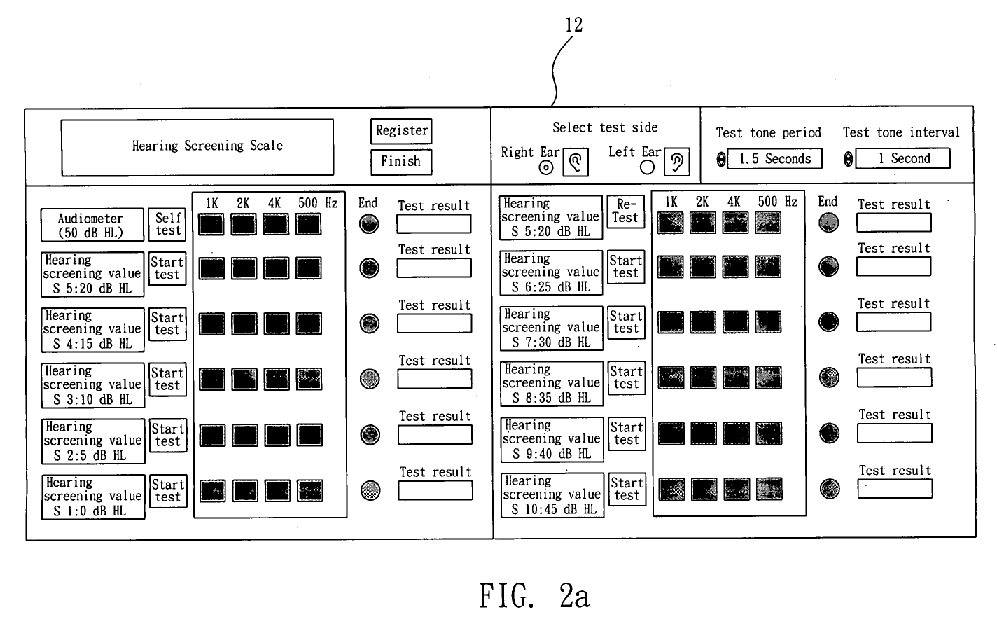

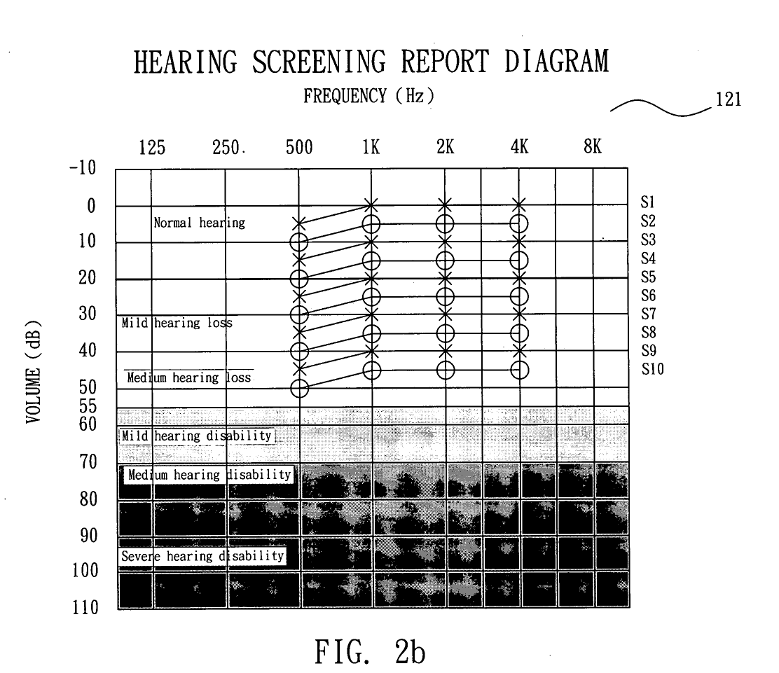

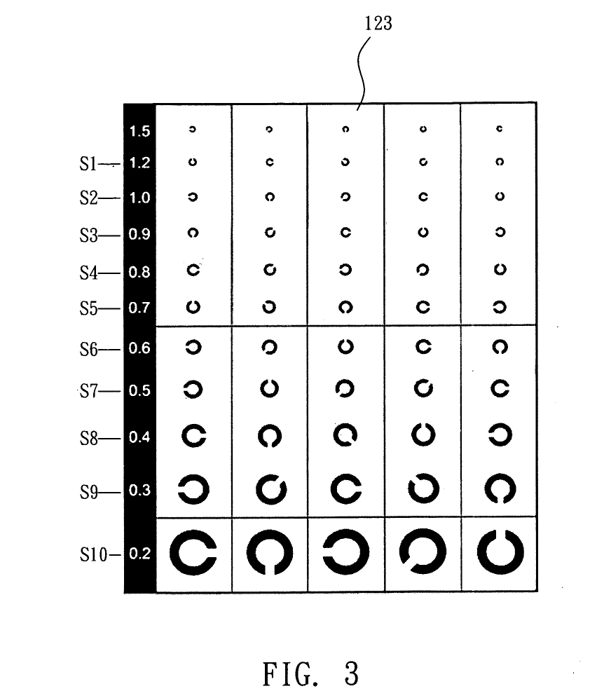

Please refer to FIGS. 1˜3, in which, FIG. 1 is a schematic drawing showing a hearing test and screening system in accordance with the present invention; FIG. 2a is a schematic drawing showing the first application program of the hearing test and screening system according to the present invention; FIG. 2b is a hearing screening report diagram of the second application program of the hearing test and screening system according to the present invention; FIG. 3 is a comparison of the hearing screening report diagram of the invention and a conventional visual acuity chart.

As illustrated, the hearing test and screening system belongs the aforesaid fourth type audiometer (screening audiometer) provided with a response button for pure tone audiometry without bone conduction threshold audiometry. The hearing screening system comprises a computer 10, an audio apparatus 20, and a pair of earphones 30.

The computer 10 is the computing and processing center of the system, comprising a graphic-control software program that comprises a first application program 11 and a second application program 12. The computer 10 can be, but not limited to, a desk computer or notebook computer. The first application program 11 and the second application program 12 are developed for applications in test and measurement subject to, for example, but not limited to, the graphical programming of Lab VIEW from National Instrument Corp., providing better human nature and interaction on the screen of the computer 10 and, allowing the user (clinical medical staff, school nurse, teacher, parent or test person) to adjust test ear, test frequency, test sound volume and other parameters through the human-machine interface screen of the computer 10 and to let the computer 10 produce a pure tone signal for screening test.

The first application program 11 is adapted for measuring the case-value of the noise floor of the test environment. During application of the first application program 11, the better ear is firstly used, assisting achievement of tolerable test environment noise floor. The second application program 12 is adapted for measuring hearing screening standard test values, including two parameters, namely, the audio frequency and the sound intensity.

The audio apparatus 20 is coupled to the computer 10 to receive the pure tone signal produced by the computer 10 and to output a set of test signals of a constant sound intensity. Each output test signal provided by the audio apparatus 20 comprises at least 3 frequencies. The set of test signals can include an audio frequency parameter and a sound intensity parameter. The audio frequency parameter can be, for example, but not limited to, 500 Hz, 1000 Hz, 2000 Hz, 4000 Hz or 6000 Hz. The sound intensity parameter can be, for example, but not limited to, a number of values selected from the group of 0 dBHL, 2.5 dBHL, 5 dBHL, 7.5 dBHL, 10 dBHL, 12.5 dBHL, 15 dBHL, 17.5 dBHL, 20 dBHL, 22.5 dBHL, 25 dBHL, 27.5 dBHL, 30 dBHL, 32.5 dBHL, 35 dBHL, 37.5 dBHL, 40 dBHL, 42.5 dBHL and 45 dBHL, i.e., the interval of every sound intensity parameter can be 2.5 dBHL, 5 dBHL or 10 dBHL. This internal can be changed subject to requirements, for example, 2.5 dBHL screening interval can be adopted for a test person which want having more precise hearing result or, 10 dBHL screening interval an be adopted for a test person which want having rough hearing result. Further, the second application program 12 can process and record the response of the test person against the audio signal 500 Hz, 1000 Hz, 2000 Hz, 4000 Hz or 6000 Hz, and display the processed result on the screen of the computer 10.

The second application program 12 can select three or four audio signals from the group of 500 Hz, 1000 Hz, 2000 Hz, 4000 Hz and 6000 Hz through a computer program-controlled method for test subject to the identity of the test person to be a young child, juvenile or adult. For example, when the test person is an ordinary young child, the second application program 12 outputs the audio signals 500 Hz, 1000 Hz, 2000 Hz and 4000 Hz with the sound intensity 20˜25 dBHL and screening interval 2.5 dBHL, 5 dBHL or 10 dBHL that may be adjusted subject to requirements. Further, the second application program 12 processes and records the responses made by the test person against the applied audio signals of 500 Hz, 1000 Hz, 2000 Hz and 4000 Hz, and then displays the processed results on the display screen of the computer 10.

When the test person is a juvenile testee, the second application program 12 outputs the audio signals 1000 Hz, 2000 Hz, 4000 Hz and 6000 Hz with the sound intensity 20˜25 dBHL and screening interval 2.5 dBHL, 5 dBHL or 10 dBHL that may be adjusted subject to requirements. Further, the second application program 12 processes and records the responses made by the test person against the applied audio signals 1000 Hz, 2000 Hz, 4000 Hz and 6000 Hz, and then display the processed result on the display screen of the computer 10.

When the test person is an adult testee, the second application program 12 outputs the audio signals 1000 Hz, 2000 Hz and 4000 Hz with the sound intensity 35˜40 dBHL and screening interval 2.5 dBHL, 5 dBHL or 10 dBHL that may be adjusted subject to requirements. Further, the second application program 12 processes and records the responses made by the test person against the applied audio signals 1000 Hz, 2000 Hz and 4000 Hz, and then displays the processed result on the display screen of the computer 10.

Further, the audio apparatus 20 can be, but not limited to, an external audio apparatus comprising a connection port 21 and a response button 22. The connection port 21 is connectable to the computer 10. The response button 22 is for enabling the user to send the feedback signal to the computer 10 for recording. The connection port 21 can be, for example, but not limited to, a USB connection port. The audio apparatus 20 can be, for example, but not limited to, a sound bluster or ivoice II.

The pair of earphones 30 is coupled to the external audio apparatus 20 for enabling the user to hear the applied audio signal. The pair of headphone 30 can be, for example, but not limited to, Telephonics THD-50P earphones or calibrated test earphones.

Further, as shown in FIG. 2b, the second application program 12 further comprises a hearing screening report diagram 121 that divides hearing acuity into Normal (S1˜S5), Hearing impaired alarm (S6˜S7) and Hearing impaired (S8˜S10).

For easy understanding of the meanings of Normal hearing (S1˜S5), Hearing impaired alarm (S6˜S7) and Hearing impaired (S8˜S10), the invention made a comparison chart between the hearing screening report diagram 121 shown in FIG. 2 and a conventional visual acuity chart 123. By means of this comparison, an ordinary test person can know his (her) hearing acuity immediately after the test as easy as a conventional visual acuity chart. As shown in FIG. 3, Normal hearing (S1˜S5) corresponds to the range 1.2˜0.7 of the visual acuity chart, Hearing impaired alarm (S6˜S7) corresponds to the range 0.6˜0.5 of the visual acuity chart, and Hearing impaired (S8˜S10) corresponds to the range 0.4˜0.2 of the visual acuity chart. When the hearing acuity of the test personal falls to the range of Hearing impaired alarm (S6˜S7) or the range of Hearing impaired (S8˜S10), the test person must see a professional doctor for further screening or treatment. By means of the aforesaid comparison chart, an ordinary test person can know one's hearing acuity immediately after the test and to receive treatment when necessary.

During screening, the test person has the earphones 30 put over his (her) head. At this time, the second application program 12 runs the hearing screening procedure through the computer 10. After installation of the program set (not shown) by the driver, the audio apparatus 20 stably outputs the selected audio signals 500 Hz, 1000 Hz, 2000 Hz, 4000 Hz or 6000 Hz to the earphones 30. When the test person hears one test audio signal, he (she) immediately clicks the response button 22, giving a feedback signal to the computer 10 for recording and then displaying it on the hearing screening report diagram 121. After repeatedly running 500 Hz, 1000 Hz, 2000 Hz, 4000 Hz or 6000 Hz audio signal hearing screening procedures, the lowest threshold value of the sound volume at every frequency, i.e., the lowest value of the sound volume hearable by the test person at every frequency. After the test, the test person can know his (her) hearing acuity through the hearing screening report diagram 121.

A person can use this test apparatus through a Windows desk computer or notebook computer having a USB interface. Because the first application program 11 and the second application program 12 are developed subject to the graphical programming of Lab VIEW, the first application program 11 and the second application program 12 provide better human nature and interaction on the screen of the computer 10 so that the user (clinical medical staff, school nurse, teacher, parent or test person) can adjust test ear, test frequency, test sound volume and other parameters through the first application program 11 and the second application program 12 on the human-machine interface screen of the computer 10 and control the computer 10 to output a pure tone signal through the external audio apparatus 20 to the earphones 30 at a constant sound volume for screening test.

The second application program 12 does not change the sound volume subject to the type of the computer 10 used, enabling the test person to hear calibrated test sounds having a standard sound volume value.

By means of the hearing test and screening system of the invention, the hearing acuity of the test person is measured accurately. Further, the operation of the hearing screening procedure is simple, shortening the hearing screening time. Further, the invention allows the test person to know the test result immediately after the test. Therefore, the invention effectively improves the drawbacks of conventional techniques.

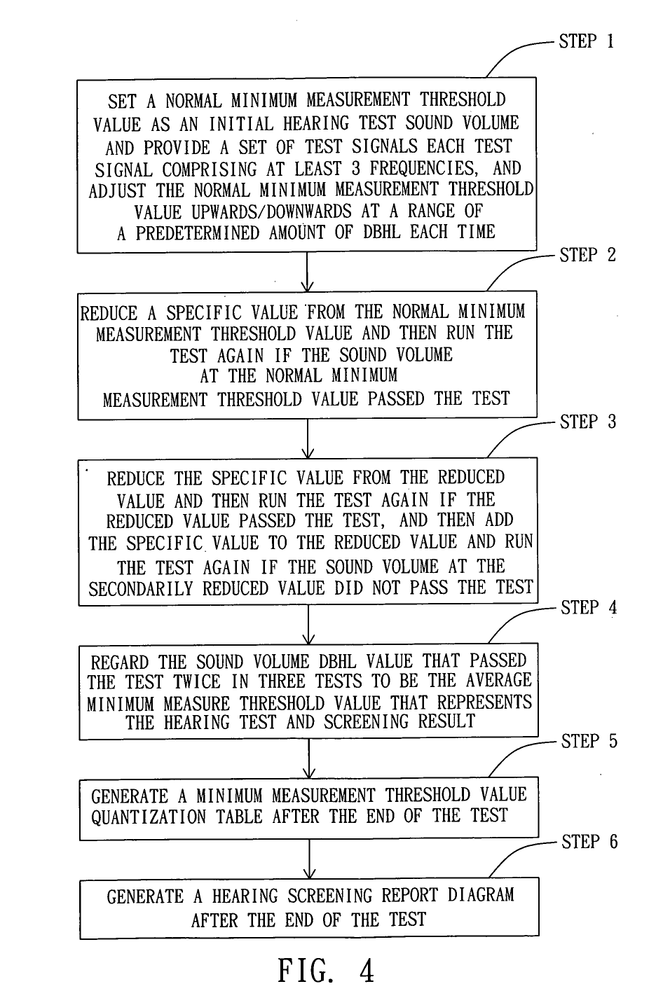

Further, the invention also provides a method for hearing threshold measurements. FIG. 4 illustrates the flow of the method for hearing threshold measurements. This method includes the steps of:

1. Set a normal minimum measurement threshold value as an initial hearing test sound volume and provide a set of test signals each test signal comprising at least 3 frequencies, and adjust the normal minimum measurement threshold value upwards/downwards at a range of a predetermined amount of dBHL each time;

2. Reduce a specific value from the normal minimum measurement threshold value and then run the test again if the sound volume at the normal minimum measurement threshold value passed the test;

3. Reduce the specific value from the reduced value and then run the test again if the reduced value passed the test, and then add the specific value to the reduced value and run the test again if the sound volume at the secondarily reduced value did not pass the test; and

4. Regard the sound volume dBHL value that passed the test twice in three tests to be the average minimum measure threshold value that represents the hearing test and screening result.

During Step 1, “Set a normal minimum measurement threshold value as an initial hearing test sound volume and provide a set of test signals each test signal comprising at least 3 frequencies, and adjust the normal minimum measurement threshold value upwards/downwards at a range of a predetermined amount of dBHL each time”, in which the normal minimum measurement threshold value can be, for example, but not limited to, 20 dBHL, and the upward or downward adjustment range per each time can be, for example, but not limited to, 2.5 dBHL, 5 dBHL or 10 dBHL.

During Step 2, “Reduce a specific value from the normal minimum measurement threshold value and then run the test again if the sound volume at the normal minimum measurement threshold value passed the test”, in which the specific value can be, for example, but not limited to, 2.5 dBHL, 5 dBHL or 10 dBHL.

During Step 4, “Regard the sound volume dBHL value that passed the test twice in three tests to be the average minimum measure threshold value that represents the hearing test and screening result”, in which the average minimum measurement threshold value is the means value of the three minimum measurement threshold values obtained from the test at 500 Hz, 1000 Hz and 2000 Hz. Alternatively, the average minimum measure threshold value can be the means value of the four minimum measurement threshold values obtained from the test at 500 Hz, 1000 Hz, 2000 Hz and 4000 Hz.

The hearing test and screening method of the invention further comprises Step 5: Generate a minimum measurement threshold value quantization table after the end of the test. This minimum measurement threshold value quantization table quantifies hearing screening results into 10 measurement threshold values (S1˜S10) subject to standards for hearing screening set by American Speech-Language-Hearing Association.

The hearing test and screening method of the invention further comprises Step 6: Generate a hearing screening report diagram 111 after the end of the test. The hearing screening report diagram 111 divides hearing acuity into Normal hearing (S1˜S5), Hearing impaired alarm (S6˜S7) and Hearing impaired (S8˜S10), as stated before.

When receiving the test, the test person has the earphones 30 put on his (her) head. At this time, the first application program runs the hearing test procedure through the computer 10. After installation of the program set (not shown) by the driver, the audio apparatus 20 stably outputs the selected audio signals 500 Hz, 1000 Hz, 2000 Hz and 4000 Hz to the earphones 30. When the test person hears one test audio signal, he (she) immediately clicks the response button 22, giving a feedback signal to the computer 10 for displaying it on the hearing screening report diagram 111. After repeatedly running 500 Hz, 1000 Hz, 2000 Hz and 4000 Hz audio signal hearing screening procedures, the average lowest measurement threshold value of the sound volume at every frequency can then be obtained, i.e., the lowest value of the sound volume hearable by the test person at every frequency. After the test, the test person can know his (her) hearing acuity through the hearing screening report diagram 111.

Thus, accurate measurement of hearing acuity can be obtained by means of the application of the hearing test and screening method of the present invention. Further, the operation of the hearing test procedure is quite simple, shortening the hearing test time. Therefore, the invention effectively improves the drawbacks of conventional hearing test techniques.

In general, the invention provides a hearing test and screening system and method having the advantages of high portability and hearing acuity measurement accuracy, simple hearing screening procedure and short hearing screening test time. Therefore, the invention effectively improves the drawbacks of conventional techniques.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A hearing test and screening system for testing and screening the hearing acuity of a person, comprising:
a computer, said computer comprising a program with graphic user interface for generating a pure tone-signal required for a hearing test;
an audio apparatus coupled to said computer and adapted for receiving said pure-tone signal from said computer and outputting a set of test signals at a constant sound volume upon receipt of said pure tone signal, each said test signal comprising at least 3 frequencies, said audio apparatus comprising a connection port connected to said computer and a response button operable by a test person to provide a feedback signal to said computer; and
a set of earphone means coupled to said audio apparatus and adapted for enabling a test person to hear said a set of test signals outputted by said audio apparatus.

2. The hearing test and screening system as claimed in claim 1, wherein said computer is selected from the group of desk computer and notebook computer; said earphone means is a calibrated test earphone.

3. The hearing test and screening system as claimed in claim 1, wherein said graphic-control program comprises a first application program and a second application program developed subject to the graphical programming of Lab VIEW from National Instrument Corp.

4. The hearing test and screening system as claimed in claim 3, wherein said first application program is adapted for measuring the case-value of the noise floor of the test environment and, during application of said first application program, the normal or better ear is firstly used, assisting achievement of tolerable test environment noise floor; said second application program is adapted for measuring hearing screening standard test values, including the parameters of audio frequency and sound intensity.

5. The hearing test and screening system as claimed in claim 1, wherein each said audio signal has an audio frequency selected from a group of 500 Hz, 1000 Hz, 2000 Hz and 4000 Hz or 6000 Hz, a sound intensity selected from a group of 0 dBHL, 2.5 dBHL, 5 dBHL, 7.5 dBHL, 10 dBHL, 12.5 dBHL, 15 dBHL, 17.5 dBHL, 20 dBHL, 22.5 dBHL, 25 dBHL, 27.5 dBHL, 30 dBHL, 32.5 dBHL, 35 dBHL, 37.5 dBHL, 40 dBHL, 42.5 dBHL and 45 dBHL; said second application program select at least three audio signals from a group of 500 Hz, 1000 Hz, 2000 Hz, 4000 Hz or 6000 Hz for test subject to the identity of the test person to be a young child, juvenile or adult.

6. The hearing test and screening system as claimed in claim 3, wherein said second application program generates a minimum measurement threshold value quantization table after the end of the test, said minimum measurement threshold value quantization table dividing the hearing screening results into 10 measurement threshold values (S1˜S10) subject to standards for hearing screening set by American Speech-Language-Hearing Association.

7. The hearing test and screening system as claimed in claim 6, wherein said 10 measurement threshold values (S1˜S10) are classified into the categories of Normal hearing (S1˜S5), Hearing impaired alarm (S6˜S7) and Hearing impaired (S8˜S10).

8. The hearing test and screening system as claimed in claim 7, wherein said Normal hearing (S1˜S5) corresponds to 1.2˜0.7 in a visual acuity chart; said Hearing impaired alarm (S6˜S7) corresponds to 0.6˜0.5 in a visual acuity chart; said Hearing impaired (S8˜S10) corresponds to 0.4˜0.2 in a visual acuity chart.

9. The hearing test and screening system as claimed in claim 1, wherein said connection port is a USB port; said audio apparatus is selected from a group of sound bluster and ivoice II.
